(19) United States
(12) Patent Application Publication
(10) Pub. No.: US 2024/0099931 A1
(43) Pub. Date: Mar. 28, 2024

(54) VAGINAL DILATOR ASSEMBLY

(71) Applicant: Helen Murphy, North Riverside, IL (US)

(72) Inventor: Helen Murphy, North Riverside, IL (US)

(21) Appl. No.: 17/951,272

(22) Filed: Sep. 23, 2022

(51) Int. Cl. A61H 19/00; A61H 23/02

(52) U.S. Cl. CPC A61H 19/44; A61H 23/02

(57) ABSTRACT

A vaginal dilator assembly for containing crystals includes a shaft having a perimeter wall enclosing a receiving space. A plurality of crystals is removably stored within the receiving space of the shaft.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to vaginal dilator devices and more particularly pertains to a new vaginal dilator device for containing crystals for combining holistic healing with dilation practice.

### (2) Description of Related Art Including Information Disclosed Under 37 CFR 1.97 and 1.98

The prior art relates to vaginal dilator devices including a variety of devices configured for facilitating in vaginal dilation. Known prior art does not allow for incorporating holistic practices.

### BRIEF SUMMARY OF THE INVENTION

An embodiment of the disclosure meets the needs presented above by generally comprising a shaft having a perimeter wall enclosing a receiving space. A plurality of crystals is removably stored within the receiving space of the shaft.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top isometric view of a vaginal dilator assembly according to an embodiment of the disclosure.

FIG. 2 is a bottom isometric view of an embodiment of the disclosure.

FIG. 3 is a cross-sectional view of an embodiment of the disclosure taken from FIG. 1 of Line 3-3.

FIG. 4 is an exploded view of an embodiment of the disclosure.

FIG. 5 is a front view of an alternative embodiment of the disclosure.

FIG. 6 is a cross-sectional view of an embodiment of the disclosure taken along Line 6-6 of FIG. 5.

### DETAILED DESCRIPTION OF THE INVENTION

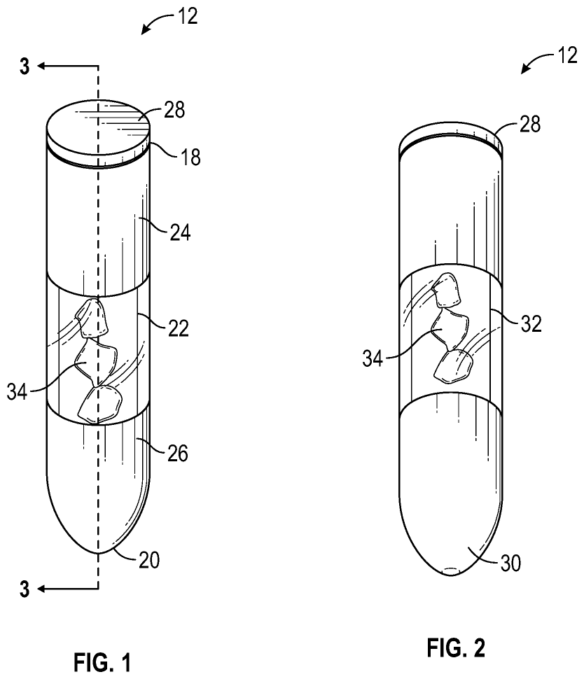

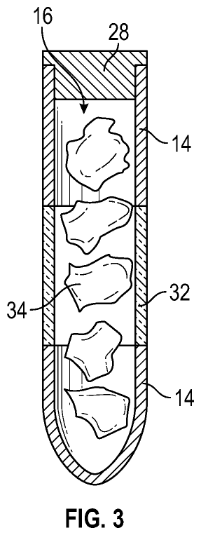

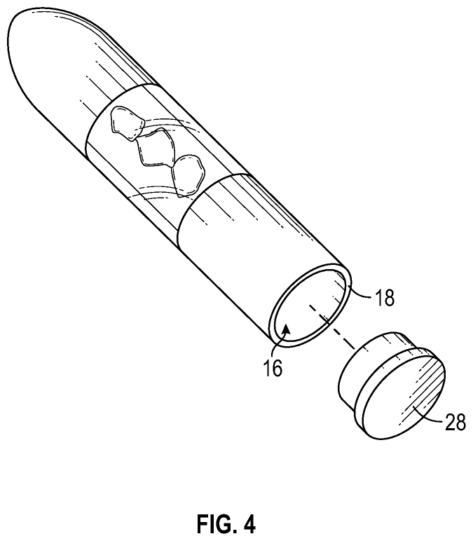

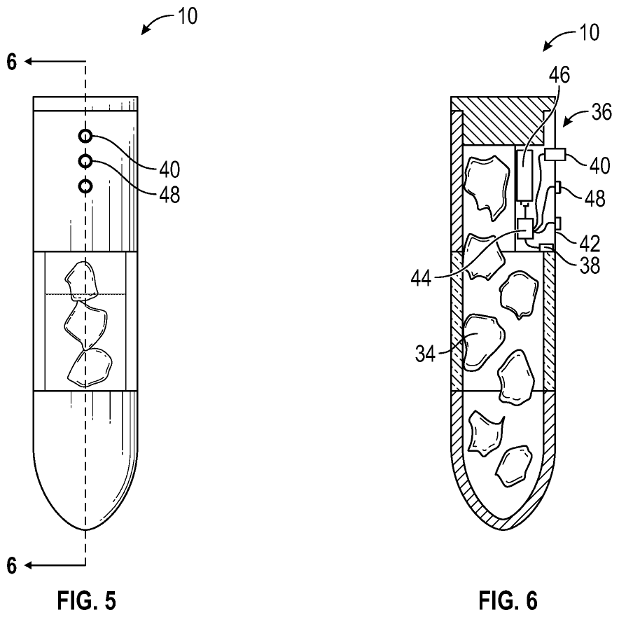

With reference now to the drawings, and in particular to FIGS. 1 through 6 thereof, a new vaginal dilator device embodying the principles and concepts of an embodiment of the disclosure and generally designated by the reference numeral 10 will be described.

As best illustrated in FIGS. 1 through 6, the vaginal dilator assembly 10 generally comprises a shaft 12 having a perimeter wall 14 enclosing a receiving space 16. The perimeter wall 14 has a first end 18, a second end 20, and a center section 22 spaced between the first 18 and second 20 ends wherein a first section 24 includes the first end 18 and a second section 26 includes the second end 20 are positioned on opposite sides of the center section 22. The first end 18 is open and may have a cap 28 removably positioned thereon for closing the first end 18. The second end 20 comprises a closed end and may have a convexly arcuate outer surface 30 to facilitate insertion of the shaft 12 into a vagina. The first 24 and second 26 sections may comprise a silicone material and the center section 22 may comprise a plastic or glass material configured for being transparent to define a window 32 into the receiving space 16. A plurality of crystals 34 is removably stored within the receiving space 16 of the shaft 12. Each of the crystals 34 are of the conventional type used in the holistic healing arts as a focus object used for meditation thus the window 32 allows the user to view the crystals 34 when using the shaft 12. In this manner, the crystals 34 help relax the user to encourage their usage of the assembly 10.

A vibrational apparatus 36 is mounted on the shaft 12 for vibrating the shaft 12. The vibrational apparatus 36 includes a vibrational motor 38 mounted in the receiving space and configured for vibrating the shaft 12 when the vibrational motor 38 is turned on. An actuator 40 is mounted on an exterior 42 of the perimeter wall 14 and is electrically coupled to the vibrational motor 38 for turning the vibrational motor 38 on or off. The actuator 40 may comprise a button or a switch.

A control circuit 44 is electrically coupled to the vibrational motor 38 and to the actuator 40. The control circuit 44 is mounted in the receiving space 16 and a power supply 46 is electrically coupled to the control circuit 44. The power supply 46 is mounted in the receiving space 16 and positioned proximate to the control circuit 44 and may comprise a battery. At least one control button 48 is mounted on the exterior 42 of the perimeter wall 14 and is electrically coupled to the control circuit 44 for controlling an intensity of vibrations imparted to the shaft 12 by the vibrational motor 38.

In use, the crystals 34 are removably stored within the receiving space 16 of the shaft 12 and are viewable through the window 32 to provide a visual focus for meditation. The second end 20 of the shaft 12 may there be inserted into the vagina or the anus of the user. The vibrational apparatus 36 facilitates in vaginal or anal dilation by actuating the vibrational motor 38 and controlling the intensity of vibrations using the control button 48. The crystals 34 may be removed and changed as desired to have the intended effect on the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A vaginal dilator assembly comprising:
a shaft having a perimeter wall enclosing a receiving space; and
a plurality of crystals being removably stored within said receiving space of said shaft.

2. The vaginal dilator assembly of claim 1, wherein said perimeter wall has a first end and a second end, said perimeter wall having a center section being spaced between said first and second ends wherein a first section includes said first end and a second section includes said second end are positioned on opposite sides of said center section.

3. The vaginal dilator assembly of claim 2, wherein said first end is open, said first end having a cap removably positioned thereon for closing said first end, said second end comprising a closed end and having a convexly arcuate outer surface.

4. The vaginal dilator assembly of claim 3, wherein said first and second sections comprises a silicone material, said center section comprising a plastic or glass material.

5. The vaginal dilator assembly of claim 1, further comprising a vibrational apparatus being mounted on said shaft and being configured for facilitating vaginal dilation by vibrating said shaft.

6. The vaginal dilator assembly of claim 5, wherein said vibrational apparatus further includes a vibrational motor being configured for vibrating said shaft when said vibrational motor is turned on, said vibrational motor being mounted in said receiving space.

7. The vaginal dilator assembly of claim 6, further including an actuator being mounted on an exterior of said perimeter wall and electrically coupled to said vibrational motor, said actuator being configured for turning the vibrational motor on or off, said actuator comprising a button.

8. The vaginal dilator assembly of claim 7, further including a control circuit being electrically coupled to said vibrational motor and to said actuator, said control circuit being mounted in said receiving space.

9. The vaginal dilator assembly of claim 8, further including a power supply being electrically coupled to said control circuit, said power supply being mounted in said receiving space, said power supply comprising a battery.

10. The vaginal dilator assembly of claim 9, further including at least one control button being mounted on said exterior of said perimeter wall and configured for controlling an intensity of vibrations imparted to said shaft by said vibrational motor, said at least one control button being electrically coupled to said control circuit.

11. A vaginal dilator assembly comprising:
a shaft having a perimeter wall enclosing a receiving space, said perimeter wall having a first end and a second end, said perimeter wall having a center section being spaced between said first and second ends wherein a first section including said first end and a second section including said second end are positioned on opposite sides of said center section, said first end being open, said first end having a cap removably positioned thereon for closing said first end, said second end comprising a closed end and having a convexly arcuate outer surface, said first and second sections comprising a silicone material, said center section comprising a plastic or glass material;
a plurality of crystals being removably stored within said receiving space of said shaft;
a vibrational apparatus being mounted on said shaft and being configured for facilitating vaginal dilation by vibrating said shaft, said vibrational apparatus further including:
a vibrational motor being configured for vibrating said shaft when said vibrational motor is turned on, said vibrational motor being mounted in said receiving space;
an actuator being mounted on an exterior of said perimeter wall and electrically coupled to said vibrational motor, said actuator being configured for turning the vibrational motor on or off, said actuator comprising a button;
a control circuit being electrically coupled to said vibrational motor and to said actuator, said control circuit being mounted in said receiving space;
a power supply being electrically coupled to said control circuit, said power supply being mounted in said receiving space, said power supply comprising a battery; and
at least one control button being mounted on said exterior of said perimeter wall and configured for controlling an intensity of vibrations imparted to said shaft by said vibrational motor, said at least one control button being electrically coupled to said control circuit.
